(19) United States
(12) Patent Application Publication
(10) Pub. No.: US 2026/0145272 A1
(43) Pub. Date: May 28, 2026

(54) TECHNIQUE FOR PREDICTING JOINING DEFECTS DURING LASER WELDING

(71) Applicant: TRUMPF Laser SE, Schramberg (DE)

(72) Inventors: Oliver Bocksrocker, Sachsenheim (DE); Jan-Patrick Hermani, Markgröningen (DE); Matthias Hartmann, Schramberg (DE); Julia Ziegler, Albstadt (DE)

(21) Appl. No.: 19/449,509

(22) Filed: Jan. 15, 2026

Related U.S. Application Data
(63) Continuation of application No. PCT/EP2024/071040, filed on Jul. 24, 2024.

(30) Foreign Application Priority Data
Jul. 27, 2023 (DE) 102023120020.8

(51) Int. Cl. B23K 26/244; B23K 26/03; B23K 26/32; G01N 21/45; G01N 33/207; G06V 10/82; G06V 20/52

(52) U.S. Cl. CPC B23K 26/244; B23K 26/032; B23K 26/32; G01N 21/45; G01N 33/207; G06V 10/82; G06V 20/52; G06V 2201/06

(57) ABSTRACT

A method for laser welding bipolar plates includes laser welding two at least partly overlapping joining partners along a specified welding path in order to produce an overlapping connection between the two joining partners, during the laser welding, observing whether, following a process zone, at least one undulating structure forms in a weld pool, based on the observing, initiating a measure for preventing or handling an incomplete connection of the two joining partners along at least one section of the welding path.

### CROSS REFERENCE TO RELATED APPLICATIONS

This application is a continuation of International Application No. PCT/EP2024/071040 (WO2025/021881A2), filed on Jul. 24, 2024, and claims benefit to German Patent Application No. DE 10 2023 120 020.8, filed on Jul. 27, 2023. The aforementioned applications are hereby incorporated by reference herein.

### FIELD

Embodiments of the present invention relate to the field of laser welding.

### BACKGROUND

Several methods for optical monitoring of welding processes are already known from the prior art. By way of example only, reference is made in this context to DE 10 2018 220 342 A1, which discloses such a monitoring method.

For the production of bipolar plates for fuel cells, metal plates or foils are welded together in pairs. At least some of the weld seams connecting the plate pairs to one another must be fluid-tight so as not to compromise the later functionality of the bipolar plate. Traditionally, a pair of bipolar plates is welded together using overlapping connections. First, the joining partners are positioned relative to one another and fixed in place. A laser welding beam then traces a specified contour on the surface of one of the joining partners, wherein the laser beam penetrates through the joining partner into the adjacent joining partner and creates a weld seam that extends at least partially into the “concealed” joining partner. However, it is possible in this case, for example due to insufficient fixation of the joining partners, that a gap is formed between the joining partners which exceeds a critical clearance and is not completely bridged by the weld seam. A person skilled in the art refers to this as a “joining defect”. When welding a bipolar plate, even a distance of 15 μm between the joining partners can lead to the formation of joining defects.

When welding overlapping connections, it is essential to avoid joining defects. One difficulty is that joining defects on the welded product are often not visible to the naked eye, as the resulting weld bead on the top side of the workpiece and the weld root on the bottom side of the workpiece often show no abnormalities. In the case of such joining defects, which are not visible from the outside, a person skilled in the art also refers to them as “false friends”.

The inspection of weld seams for joining defects is therefore time-consuming and costly.

### SUMMARY

Embodiments of the present invention provide a method for laser welding bipolar plates. The method includes laser welding two at least partly overlapping joining partners along a specified welding path in order to produce an overlapping connection between the two joining partners, during the laser welding, observing whether, following a process zone, at least one undulating structure forms in a weld pool, based on the observing, initiating a measure for preventing or handling an incomplete connection of the two joining partners along at least one section of the welding path.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic top view of a bipolar plate with the process zone drawn and the surrounding weld pool of a laser welding process according to some embodiments;

FIG. 2a and FIG. 2b show a schematic representation of an overlapping connection between two joining partners in a sectional view in each case, wherein FIG. 2b shows the defect image of an incomplete connection (joining defect) according to some embodiments;

FIG. 3a, and FIG. 3b show a schematic representation of the geometry of a weld pool during the creation of an overlapping connection between two joining partners by means of laser welding according to some embodiments;

FIG. 4 shows an image of a weld seam with joining defects, illuminated from the side; and

FIG. 5a. FIG. 5b and FIG. 5c show process emissions of a laser welding process, recorded at various points in time during the creation of the weld seam shown in FIG. 4 according to some embodiments.

### DETAILED DESCRIPTION

Embodiments of the present invention can improve reliability with regard to tightness when welding overlapping connections. In particular, it is to be made possible to make a reliable assessment of the formation of joining defects already during the welding process in order to derive appropriate measures.

According to a first aspect, a method for laser welding bipolar plates is provided. The method comprises laser welding two at least partly overlapping joining partners along a specified welding path in order to produce an overlapping connection between the joining partners.

In the context of the present disclosure, an overlapping connection is to be understood as a welded joint in which the weld seam extends completely across the thickness of a first of the joining partners (=upper joining partner) and at least partly into the second (lower) joining partner. For this purpose, the laser beam is directed along the welding path onto a surface of the first joining partner and melts the joining partners into the second joining partner or even across the entire thickness of both joining partners. In this context, the laser welding is preferably carried out in a deep welding mode, in which the laser beam forms a vapor capillary in the process zone where it interacts with the joining partners, wherein this vapor capillary extends deep into the joining partners and promotes an efficient welding process. The welding process is preferably conducted in a computer-controlled manner.

For the laser welding process, a solid-state laser (e.g., a disc laser or fiber laser) or a diode laser can be used, for example. For example, a single-mode laser with a laser power of 500 W to 2 kW or a multi-mode laser with a laser power of 2 kW to 5 kW can be used. A preferred power output of the laser processing beam when welding bipolar plates can be in the range of 10 W to 2000 W, in particular from 50 W to 700 W. When using a single-mode laser, the laser beam can have a beam parameter product in the range of 0.36 mm*mrad to 16 mm*mrad, in particular of approximately 0.6 mm*mrad. When using a multi-mode laser, the laser beam can have a beam parameter product of at least 3 mm*mrad. In particular, an infrared laser with a wavelength in the range from 800 nm to 1200 nm, in particular 1030 nm or 1070 nm, can be used as the laser. Alternatively, a VIS laser with a wavelength in the blue spectral range of, for example, 400 nm to 450 nm, or in the green spectral range, in particular with 515 nm, can be used as the laser.

The (focused) processing laser beam can have a beam diameter in the range of 10 μm to 300 μm, in particular in the range of 30 μm to 70 μm (single-mode) or in the range of 50 μm to 170 μm (multi-mode), in the region of the plane of the workpiece surface, i.e., the surface of the upper joining partner.

A feed rate at which the laser beam is moved relative to the workpiece surface along the welding path can be in the range of 100 mm/s to 5000 mm/s, in particular in the range of 300 mm/s to 2000 mm/s.

A processing optical unit through which the laser beam is directed onto the workpiece and, in particular, focused, can have an imaging ratio of 1:1 to 5:1, preferably 1.5:1 to 2:1.

The method according to embodiments of the invention further comprises observing, during the welding process, whether following the process zone at least one undulating structure forms in the weld pool, which preferably extends transversely to the feed direction.

The process zone refers to the region in which the processing laser beam interacts with at least one of the joining partners during the welding process. When deep welding, the process zone is usually characterized by the formation of a vapor capillary that substantially extends over the entire depth of the later weld seam. The material of the joining partners is melted in and around the process zone. The relative feed movement of the laser beam relative to the workpiece surface along the welding path creates an elongated weld pool. The temperature in the weld pool decreases with distance from the process zone. Following the process zone, the material of the joining partners initially remains molten before solidifying into the weld seam. In this region between the process zone and the already solidified weld seam, changes in the weld pool dynamics may occur during the welding process that are visually perceptible. In the present case, the inventors have recognized that an undulating structure forms on the surface of the weld pool following the process zone immediately before a joining defect is formed. The term “undulating structure” refers here to at least one elevation or depression of the weld pool surface. This at least one elevation or depression can in particular have an elongated extension which is substantially aligned transversely to the feed direction or to the length of the weld pool. The observation of changes in the weld pool can preferably be carried out on the basis of sequentially recorded individual images of the weld pool. This allows the change to be detected during the welding process.

The structural change in the weld pool typically manifests itself in a local increase or decrease in process emissions (or process radiation) in the region of the weld pool. This can be visually detected.

The method according to embodiments of the invention further comprises initiating, in response to the observation, a measure for preventing or handling an incomplete connection of the joining partners along at least one section of the welding path.

In this context, an “incomplete connection” is to be understood as synonymous with a joining defect. According to the observations of the inventors, a structural change following the process zone is already visible in the weld pool during the welding process when an incomplete joint (i.e., an untight spot) begins to form. At the beginning of the detection of a corresponding change in the weld pool dynamics, a joining defect has therefore not necessarily occurred yet. However, if the welding process continues unchanged, a joining defect is likely to occur. Depending on the situation, measures to prevent or handle joining defects can be initiated by detecting structural changes in the weld pool.

One measure to prevent an incomplete connection can, for example, involve changing the welding parameters so that the welding process can continue without joining defects occurring. Alternatively or additionally, the welding process can be interrupted and continued with changed parameters, wherein the fixation can also be improved in order to prevent a gap between the joining partners. One measure for handling an incomplete connection can comprise, for example, welding the welding path again, or classifying the welded workpiece as scrap, or reworking the fixation of the joining partners in the region of the suspected joining defect. Improvements to the fixation of the joining partners can comprise, in particular, the correction or supplementation of the clamping positions at which the joining partners are pressed against one another with appropriate clamping means.

The processing of observations during the welding process according to embodiments of the invention allows components (in particular bipolar plates) which are highly unlikely to meet the requirements for weld seam tightness to be efficiently sorted out. Furthermore, the formation of joining defects can be prevented either during the welding process or for subsequent welding processes (for example, by improving the fixation of the joining partners), thereby improving the overall welding result.

When observing the weld pool following the process zone, it can also be ascertained that the weld pool extends. An extension of the weld pool following the process zone can be used as a further indication of the formation of a joining defect.

Preferably, each of the joining partners can consist of a metallic material and have a thickness in the range from 5 μm to 500 μm, in particular in the range from 50 μm to 300 μm. The joining partners can typically be metallic foils based on iron, copper, or aluminum. For example, each of the joining partners can be made of stainless steel, for example of type 1.4404. For example, a bipolar plate for use in a fuel cell can be produced by welding the joining partners together. However, the application of embodiments of the invention is not limited to the production of bipolar plates.

The method according to embodiments of the invention can further comprise determining a position of the observed change in the weld pool along the welding path. To this end, the time elapsed since the start of the welding process can be continuously acquired while observing the weld pool. By comparing the feed rate of the welding process and the geometry of the welding path (both of which are known), the position of the weld pool change on the welding path can be determined. This position then indicates the occurrence (or approximate start) of a joining defect along the weld seam being formed. Knowing the position at which the weld pool change occurred allows targeted measures to be taken to handle (suspected) joining defects. For example, the clamping positions at which two joining partners are fixed relative to one another can be corrected and/or supplemented in order to prevent the formation of critical clearances between the joining partners. For example, this allows any geometric peculiarities (e.g., distortion) of the joining partners to be taken into account for a batch of joining partners to be welded. Alternatively or additionally, welding parameters can be adjusted at the “critical” positions along the welding path in order to counteract the formation of joining defects. Process adjustments can be particularly helpful if joining defects always occur in the same places during multiple consecutive welds of a batch.

According to a further aspect of the invention, a system for laser welding is provided. The system comprises a laser welding system for laser welding two at least partly overlapping joining partners along a specified welding path in order to produce an overlapping connection between the joining partners. The system further comprises an observation device for optically monitoring the welding process, wherein the observation device is designed to detect the formation of undulating structures in the weld pool following the process zone. The system further comprises a control device that is designed to initiate a measure for preventing or handling an incomplete connection of the joining partners along at least one section of the welding path when the observation device detects the formation of at least one wave in the weld pool following the process zone.

The laser welding system can be a conventional laser welding system suitable for welding bipolar plates. At the time of this application, the applicant is marketing various versions of such laser welding systems. The control device can, in particular, be a computer which is designed to control the laser welding system. The processing of the optical signals captured by the observation device and the control of the overall process can be carried out either on separate computers (or computing devices) or on a shared computer (in particular the control device) for controlling the entire system.

The observation device can comprise, for example, a camera. The camera can use a CMOS sensor or a CCD sensor, for example, or an InGaAs-based sensor (InGaAs=indium gallium arsenide). The exposure time of the camera can be in the range from 1 μs to 20,000 μm, in particular in the range from 1 μs to 1000 μs. A capture rate of the camera can be at least 100 Hz, in particular at least 1000 Hz. The camera can be designed in particular for observing wavelengths in the range from 300 nm to 2000 nm, in particular in the range from 600 nm to 1000 nm. The wavelength of the processing laser beam, which can be 1030 nm or 1070 nm, for example, should not be detectable by the camera. For example, the camera can use a bandpass filter for a wavelength range from 600 nm to 1000 nm, or a broadband filter with a spectral width of at least 200 nm, wherein the wavelengths of the processing laser beam (e.g., 1030 nm or 1070 nm) are blocked. One advantage of using a bandpass filter is good contrast.

The camera can preferably be aligned with the workpiece surface through the beam path of the processing laser beam, in particular coaxially or with an angular offset of up to 15% to the processing laser beam. In particular, the field of view of the camera can be coupled into the beam path of the processing laser beam via a partially transparent mirror. By integrating the observation device into the laser welding device in this manner, it is possible to ensure that the observation device is precisely aligned with the process zone or the weld pool at every stage of the process.

One advantage of using a camera as an observation device is that it can be integrated relatively easily into the processing optical unit (substantially) coaxially with the laser beam.

In addition to or as an alternative to a camera, the observation device can comprise at least two photodiodes having different measuring positions. In particular, at least two photodiodes can each be aligned with a measuring position offset from one another in the feed direction following the weld pool. The wave formation in the weld pool can be inferred from the difference in intensity of the process emission detected by each of the photodiodes. One advantage of using photodiodes is the lower data rate and higher sampling rate compared to using a camera.

In addition or alternatively, the observation device can comprise an optical coherence tomography (OCT) device. As an alternative to OCT, a laser interferometer can also be used. The OCT or the laser interferometer has a spatial resolution in the welding direction following the process zone. One advantage of using an OCT is its higher resolution compared to the use of photodiodes.

A combination of several of the aforementioned and/or further detection means in the observation device is possible. By combining them, the advantages of the individual detection means can be combined in order to achieve even better observation results.

Preferably, the observation device does not comprise any external lighting. Lighting is possible and can be used in accordance with alternative variants. However, by deliberately refraining from providing the observation device with a lighting solution, better detection of process emissions can be achieved. The reflection of the lighting would overlap with the process emissions and possibly make it more difficult to detect the structural changes following the weld pool.

The observation device can further comprise an evaluation unit that uses a neural network trained to recognize waves (i.e., undulating structures) in the weld pool. The neural network can, in particular, be a convolutional neural network (CNN) that is qualified for spatial resolution (≥2 pixels). The neural network can have a U-Net architecture and evaluate the optical signals provided by the observation device based on semantic segmentation. By using a neural network, the reliability and efficiency of wave structure detection can be improved.

According to a further aspect of the invention, a computer program product is provided which contains computer-readable instructions for carrying out a method according to any one of the variants described above in a laser welding system according to any one of the variants described above. The computer program product can, for example, be executed on the control device of a laser welding system according to embodiments of the invention.

Identical or functionally identical elements are provided with the same reference signs in the figures.

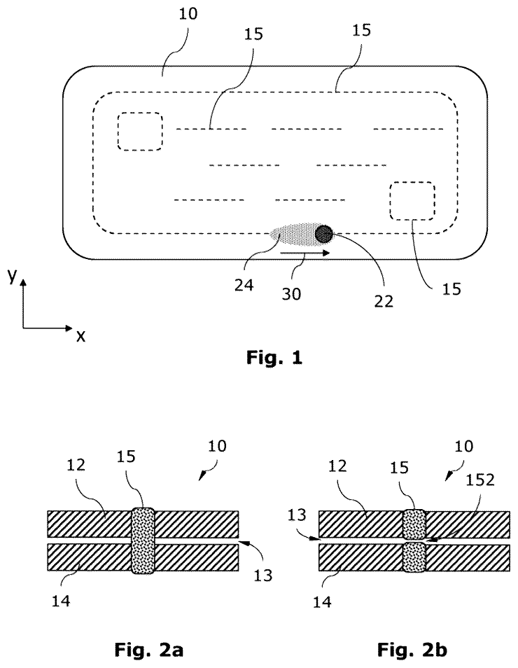

FIG. 1 shows a schematic top view of a bipolar plate 10. The bipolar plate 10 substantially consists of two flat, structured joining partners which—basically acting as respective half-shells of the bipolar plate 10—are connected to one another by means of several weld seams 15 and form fluid channels (not shown in FIG. 1) inside of the bipolar plate 10. The half-shells of the bipolar plate 10 are welded together by means of laser welding. To this end, the two joining partners are positioned and fixed precisely in relation to one another, and a processing laser beam is directed in a feed direction 30 along a welding path (corresponding to the contour of the weld seams 15 shown) onto the upper one of the joining partners in order to create an overlapping connection. In the process, the material of the joining partners is liquefied by the action of the laser beam in a process zone 22, mixes in a common weld pool 24 following the process zone, and finally solidifies while forming the weld seam 15.

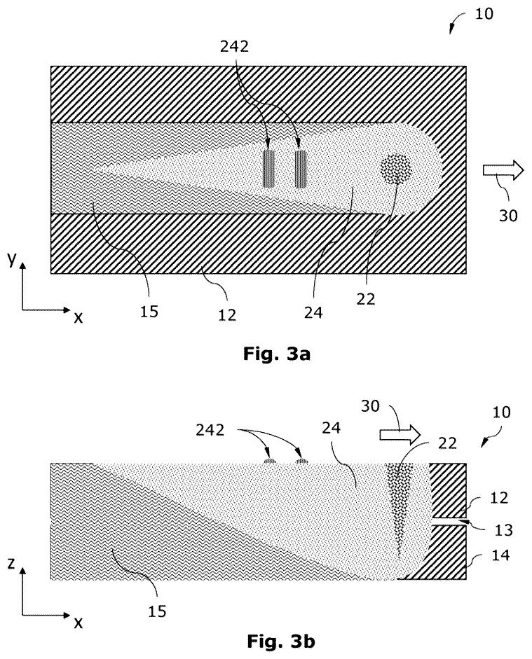

FIGS. 2a and 2b each show an overlapping connection between a first joining partner 12 and a second joining partner 14 of a component 10 (e.g., a bipolar plate). In FIG. 2a, the joining partners 12, 14 are connected to one another by a weld seam 15. According to FIG. 2b, the weld seam 15 extends through both joining partners 12, 14. However, a gap 13 between the joining partners 12, 14 is not completely bridged by the weld seam 15. The defect 152 in the weld seam 15 according to FIG. 2b results in an incomplete connection (joining defect) between the joining partners 12, 14. The connection is not tight at the position of the joining defect 152. From the outside, the defect 152 is not visible on the component 10, as the top and bottom of the weld seam 15 do not show any suspicious irregularities.

FIGS. 3a and 3b schematically show the geometry of a weld pool 24 during laser (beam) welding. FIG. 3a shows a sectional top view of a workpiece 10 or of the upper joining partner 12 of the workpiece 10 facing the used laser welding optical unit. FIG. 3b shows the same weld pool 24 in a sectional side view extending parallel to the feed direction 30 of the processing laser beam. During laser welding, the material of the joining partners 12, 14 is heated to a high temperature in the process zone 22, where the processing laser beam interacts directly with the workpiece. During deep welding, a vapor capillary forms in the process zone 22, extending over a large part of the welding depth. The material of the joining partners 12, 14 is melted around the vapor capillary. In the weld pool 24, the material of both joining partners 12, 14 mixes and solidifies following the process zone 22 to form the joint weld seam 15. When creating such overlapping connections, it is possible—for example, if a gap 13 between the joining partners 12, 14 exceeds a critical dimension—that the weld seam 15 created does not completely bridge the gap 13 between the joining partners 12, 14, resulting in the partial formation of joining defects 152. The inventors of the present invention have ascertained that, immediately before the formation of joining defects 152 during the welding process, at least one undulating structure 242 is formed on the surface of the weld pool 15 following the process zone 22. The undulating structure 242 is generally oriented perpendicular to the feed direction 30. Based on this knowledge, it is possible to take measures to prevent or handle a joining defect 152 during the welding process.

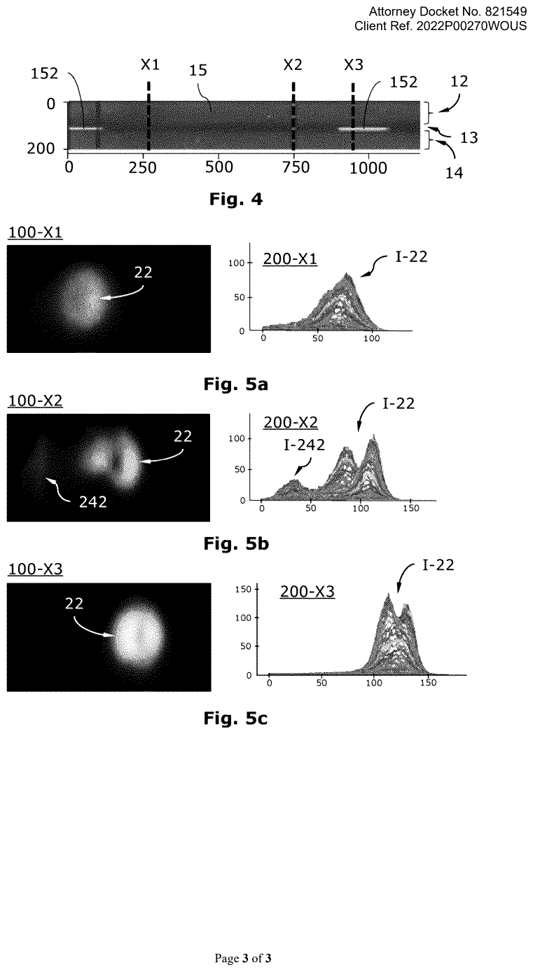

FIG. 4 shows an image of a weld seam 15, illuminated from the side. The image shows the regions of the lower (second) joining partner 14 and the upper (first) joining partner 12, which are separated from one another by a narrow gap region (see 13). At the level of the gap 13, bright lines can partly be seen, each of which indicates a joining defect 152 in the weld seam 15. The weld seam 15 shown extends through the joining partners 12, 14 of a bipolar plate and was created at a feed rate of 500 mm/s with a laser power of 200 W.

FIGS. 5a, 5b, and 5c each show a camera image 100-X1, 100-X2, 100-X3 of the weld pool 22 together with the process zone 24 during the welding process for creating the weld seam 15 shown in FIG. 4. The images were captured via the beam path of the processing laser beam—i.e., perpendicular or substantially perpendicular to the surface of the upper joining partner 14—without additional lighting. In the images 100-X1, 100-X2, and 100-X3, the process emissions at determined points in time X1, X2, and X3 during the welding process can be seen as bright regions in each case. In addition, the diagrams 200-X1, 200-X2, and 200-X3 each show sets of curves which indicate the intensity of process emissions in the observed region. The curves with the greatest deflection show the intensity along a line that extends centrally through the weld pool 24 in the feed direction, where the greatest process emissions occur, in each case. The flatter lines of the sets of curves indicate intensity gradients in the edge regions of the weld pool 24 in each case.

FIG. 5a shows the process emissions during the laser welding process at the point in time corresponding to position X1 of the weld seam 15 in FIG. 4. There is no joining defect 152 at the position X1 (see FIG. 4). The bright region in the image 100-X1 shows the process emissions in the process zone 22, which are also reflected in the intensity curves I-22 in the diagram 200-X1.

FIG. 5b shows the process emissions in relation to position X2 of the weld seam 15 (see FIG. 4), i.e., shortly before the formation of a joining defect 152. The image 100-X2 shows, on the one hand, the process emissions of the process zone 22 in the weld pool 15, with the keyhole clearly visible in the center as a gap in intensity. On the other hand, the image 100-X2 shows a further bright region 242 following the process zone 22. In the diagram 200-X2, this region is shown by the deflection of the intensity curves I-242. The shape of the process emissions following the process zone 22 results from a wave 242 that is formed in the weld pool 22 during the welding process.

Finally, FIG. 5c shows the process emissions in relation to the position X3 of the weld seam 15 (see FIG. 4), where a joining defect 152 is present. The image 100-X3 and the corresponding intensity curve 200-X3 again show high process emissions in the region of the process zone 22 with a recognizable keyhole (see also I-22 in the diagram 200-X3). No further intensity peaks can be detected following the process zone.

According to the observations of the inventors, the irregularities in the weld pool 24 shown in FIG. 5b were, in each case, only visible shortly before the formation or at the beginning of a subsequent joining defect 152. Any irregularities in process emissions in the region of the process zone 22 did not allow any conclusions to be drawn regarding the occurrence or risk of joining defects.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for laser welding bipolar plates, the method comprising:
laser welding two at least partly overlapping joining partners along a specified welding path in order to produce an overlapping connection between the two joining partners;
during the laser welding, observing whether, following a process zone, at least one undulating structure forms in a weld pool;
based on the observing, initiating a measure for preventing or handling an incomplete connection of the two joining partners along at least one section of the welding path.

2. The method according to claim 1, further comprising observing that the weld pool extends following the process zone.

3. The method according to claim 1, wherein each of the two joining partners comprises a metallic material and has a thickness in a range from 5 μm to 500 μm.

4. The method according to claim 1, further comprising determining a position of a change in the weld pool along the welding path.

5. A system for laser welding, the system comprising:
a laser welding system for laser welding two at least partly overlapping joining partners along a specified welding path in order to produce an overlapping connection between the two joining partners;
an observation device for optically monitoring the laser welding, wherein the observation device is configured to detect formation of undulating structures in a weld pool following a process zone; and
a control device configured to initiate a measure for preventing or handling an incomplete connection of the two joining partners along at least one section of the welding path when the observation device detects the formation of at least one wave in the weld pool following the process zone.

6. The system according to claim 5, wherein the observation device comprises a camera.

7. The system according to claim 5, wherein the observation device comprises at least two photodiodes having different measurement positions.

8. The system according to claim 5, wherein the observation device comprises an optical coherence tomography device.

9. The system according to claim 5, wherein the observation device comprises an evaluation unit that uses a neural network trained to recognize waves in the weld pool.

10. A non-transitory computer-readable medium having computer-readable instructions stored thereon, the computer-readable instructions, when executed by a computer processor, causing performance of a method according to claim 1.
